(19) United States
(12) Patent Application Publication
(10) Pub. No.: US 2021/0177664 A1
(43) Pub. Date: Jun. 17, 2021

(54) Method and Apparatus for a Perforable Absorbent Portion Bandage (PAP-B) for Avoidance of Constant Adhesive Removal

(71) Applicant: Fei Mei Chan, New York, NY (US)

(72) Inventor: Fei Mei Chan, New York, NY (US)

(21) Appl. No.: 16/710,977

(22) Filed: Dec. 11, 2019

(51) Int. Cl. A61F 13/02; A61F 13/00; A61M 25/02; A61L 15/24; A61L 15/58

(52) U.S. Cl. CPC A61F 13/0266; A61F 13/0206; A61F 13/00063; A61M 25/02; A61F 13/0213; A61M 2025/0273; A61F 13/0253; A61L 15/24; A61L 15/58; A61F 2013/00293; A61F 13/0226

(57) ABSTRACT

An apparatus for removing a dressing without removing an adhesive portion comprising: an absorbent central portion; an adhesive frame; the absorbent portion coupled to the adhesive frame by a perforated border; and the perforated border with a tab, whereby a user holds the tab and pulls in any one of a direction to loosen free the absorbent portion from the adhesive frame via the perforated border, thereby freeing the absorbent portion from an applied skin surface without a need for removing the adhesive frame from the applied skin surface for mitigating against a risk of MARSI, tube dislodgement, or any risk associated with constant adhesive tape removal from a skin surface during long-term wound or tube insertion dressing.

### FIELD OF INVENTION

The invention pertains generally to the field of wound or insertion site dressings, and more particularly to a dressing with a perforable absorbent portion, avoiding constant removal of an adhesive from an applied skin surface.

### BACKGROUND OF INVENTION

One of the most pervasive—yet largely unacknowledged—medical conditions seen within any care setting and amongst any age group is a condition known as Medical-Adhesive Related Skin Injury, or MARSI. While a convenient acronym, it is not so convenient for those affected by the condition. For those susceptible, MARSI is manifested as an erythema, trauma, or other reaction, including formation of vesicles, skin erosion, epidermal tears, or bulla, just shortly after removal of an adhesive from a wound or insertion site-adjacent skin surface. In short and in simple terms, MARSI is caused by constant exposure and removal of an adhesive from a skin surface of a dermatologically susceptible patient. As earlier stated, it is blind to patient age and standard of patient care or setting, and for those roughly 20% of adhesive bandage-clad patients inflicted with MARSI, it deeply impacts their health, safety, and above all else, quality of life. Suffice it to say, adding yet another medical complication onto the bevy of conditions that the patient is already being treated for does not help in containing spiraling health care costs.

More specifically, there are a number of different types of MARSI, each exhibiting their own specific symptom or indication. Whether MARSI dermatitis, maceration, or folliculitis, they all share a common symptom: Pain and irritation to a patient, whom in all likelihood, is already managing the pain and discomfort from the condition the initial treatment was sought after. MARSI dermatitis is characterized by skin redness with clearly defined borders, and in certain cases, can result in an allergic reaction in which vesicles appear for weeks on-end after removal of the adhesive. MARSI maceration is far easier to detect and diagnose due to the appearance of greyness or whiteness of the skin after adhesive removal. Additionally, wrinkling of the adhesive-exposed surface of the skin occurs, further risking the patient for tearing, abrasion, or rupturing the skin, leading to infections. Finally, MARSI folliculitis is characterized by pustules or papules formed at the hair follicles residing on the adhesive-exposed surface of the skin.

The etiology underlying MARSI is understood as being due to a stronger adhesion between skin-adhesive layer compared to skin-skin and the subsequent mechanical pulling of the skin-adhesive layer from underlying skin layers resulting in the removal of the epidermis from the dermis. The exposed dermis alters the patients skin barrier function, resulting in rendering the patient prone to the infections and immunological responses underlying the symptoms and indications described above. This removal of epidermis and exposure of dermis is further exacerbated during constant mechanical pulling of the adhesive layer or skin-adhesive layer as a result of constant re-bandaging or re-dressing required with patients experiencing excess exudate or discharge. Another risk factor not mentioned is the risk of dislodging a tube insertion as a result of the constant mechanical pulling.

As with other pathologies, prevention is the best treatment. However, in the case of MARSI, complete prevention is all but futile, with practitioners and care givers resigned to mitigation and crossed fingers. Mitigation comes in the form of using foam and silicone tape, such as 3M's Medipore tape, which are softer and gentler on the skin. Additionally, removing the adhesive slowly in the direction of hair growth and the tape parallel with the skin and pushing the skin away from the tape with the other hand is yet another mitigating factor. However, neither of these mitigating factors or products in the market address the broader underlying issue, which is the constant removal of an adhesive due to re-bandaging/dressing to address excess exudate/discharge.

Despite the risk posed by repeated pulling of an adhesive, there is currently a void in the market and prior art for outright prevention of this risk. Thus, there is a need for a bandage/dressing apparatus and method for preventing the risk of MARSI by minimizing the occurrences of adhesive removal from a wound, tube-insertion site, or adjacent site. It is blatantly evident that mere proper technique of application and removal of adhesives with the current products in the market are inadequate in addressing this growing medical concern—costing the well-being and quality of life for patients and their loved ones alike, not to mention the overall resource and financial burden on the healthcare system.

### SUMMARY

The invention resides in enabling a healthcare provider or loved one to avoid the constant or repeated removal of an adhesive layer on an applied skin surface, thereby preventing the mechanical pulling of the superficial epidermal layer, exposing the dermis to risk of MARSI. Advantageously, the avoidance of constant pulling of an adhesive layer prevents the risk of tube dislodgement during catheterization and the like.

It is one object of the present disclosure to provide an apparatus for removing a dressing or bandage without removing an adhesive portion comprising: an absorbent portion; an adhesive frame; the absorbent portion coupled to the adhesive frame by a perforated border (perforable absorbent portion bandage—PAP-B); and the perforated border with a loose or hanging tab, whereby a user holds the tab and pulls in any one of a direction of any one of an edge of the perforated border to loosen free the absorbent portion from the adhesive frame, thereby freeing the absorbent portion from an applied skin surface without a need for removing the adhesive frame from the applied skin surface.

It is another object of the present disclosure to provide for an apparatus for dressing a wound comprising: an absorbent central portion; an adhesive frame; and the absorbent central portion perforably coupled to the adhesive frame (PAP-B), wherein the adhesive frame is further adhered to any one of a foundation layer with a central opening therebetween, wherein a bottom surface of the adhesive frame is adhered to a top surface of the foundation and a bottom surface of the foundation is applied to and adhered onto an applied skin surface. Preferably, the foundation would have a square or circle shape with a center aperture/opening to accommodate a PAP-B over a wound, tube-insertion site, or adjacent site.

It is yet another object to dispose a center aperture or opening on the PAP-B, along with a slit opening along any one of an edge of the PAP-B, running through-line into the interior and terminating into the center aperture/opening in order to accommodate a tube insertion. Likewise, the foundation layer may also be disposed with a slit opening along any one of an edge of the foundation, running through-line across any one of the frames or borders of the foundation and terminating into the center aperture/opening of the foundation in order to accommodate a tube insertion.

Finally, it is yet another object to provide for a method for applying and removing a dressing without removing an adhesive portion comprising the steps of: firstly, coupling an absorbent central portion to an adhesive frame by a perforated border; secondly, holding a tab disposed at any point of the perforated border, whereby a user holds the tab; thirdly, pulling the tab in any one of a direction of an edge of the perforated border to loosen free the absorbent portion from the adhesive frame; fourthly, applying at least a second adhesive frame coupled to the absorbent portion onto a previously applied first adhesive frame top surface, thereby not requiring constant removal of the adhesive frame from the applied skin surface; and finally, removing only a last applied absorbent portion and a first applied adhesive frame from the applied skin surface once wound or insertion site dressing is no longer required.

### BRIEF DESCRIPTION OF FIGURES

The drawings illustrate the design and utility of embodiments of the present invention, in which similar elements are referred to by common reference numerals. In order to better appreciate the advantages and objects of the embodiments of the present invention, reference should be made to the accompanying drawings that illustrate these embodiments.

However, the drawings depict only some embodiments of the invention, and should not be taken as limiting its scope. With this caveat, embodiments of the invention will be described and explained with additional specificity and detail through the use of the accompanying drawings in which:

FIG. 1 depicts a top-down view of the adhesive frame perforably coupled to the central absorbent portion in accordance with an aspect of the invention.

FIG. 2 depicts a close-up view of the perforated border and tab in accordance with an aspect of the invention.

FIG. 3 depicts a side view of a plurality of adhesive frames stacked on top of one another in accordance with an aspect of the invention.

FIG. 4 depicts a top-down view of the adhesive frame coupled to the absorbent portion with a slit opening terminating in a central aperture to accommodate a tube insertion in accordance with an aspect of the invention.

FIG. 5 depicts a top-down view of the adhesive frame coupled to the absorbent portion along side a foundation layer in accordance with an aspect of the invention.

FIG. 6 depicts a top-down view of the adhesive frame coupled to the absorbent portion adhered onto a foundation layer in accordance with an aspect of the invention.

FIG. 7 depicts a method flow diagram in accordance with an aspect of the invention

### DETAILED DESCRIPTION

In the following description, for purposes of explanation, numerous specific details are set forth in order to provide a thorough understanding of the invention. It will be apparent, however, to one skilled in the art that the invention can be practiced without these specific details.

Reference in this specification to “one embodiment” or “an embodiment” means that a particular feature, structure, or characteristic described in connection with the embodiment is included in at least one embodiment of the invention. The appearances of the phrase “in one embodiment” in various places in the specification are not necessarily all referring to the same embodiment, nor are separate or alternative embodiments mutually exclusive of other embodiments. Moreover, various features are described which may be exhibited by some embodiments and not by others. Similarly, various requirements are described which may be requirements for some embodiments but not other embodiments.

The present embodiments disclose apparatus and methods for allowing one to perforably-remove an absorption layer without a constant removal of an adhesive frame.

The claimed invention eliminates the need for constant removal of an adhesive during re-bandaging, and as a result, preventing the risk of MARSI and other dermatological conditions alike, not to mention the risk of tube dislodgement. The claimed invention allows for a user to hold a tab and pull in any one of a direction of any one of an edge of the perforated border to loosen free the absorbent portion from the adhesive frame, thereby freeing the absorbent portion from an applied skin surface without a need for removing the adhesive frame from the applied skin surface for mitigating against a risk of MARSI, tube dislodgement, or any risk associated with constant adhesive tape removal from a skin surface during long-term wound or tube insertion dressing.

Constant removal is especially a concern for patients with embedded percutaneous devices, such as catheters, whereby excess fluid or leakage persists. In such patients, prolonged exposure to a soaked dressing (due to leakage) that rests directly on skin, coupled with frequent dressing changes, result in skin irritation, and in severe cases, open sores and wounds. Severity of damage to the skin gets progressively worse as the duration of leakage exposure persists and the frequency of dressing changes increases.

For these patients, it is a constant trade-off between changing the dressing (and thus causing the patient discomfort and skin irritation) versus leaving the soiled dressing on a patient's skin (which also causes irritation, particularly if the leakage is acidic, as is the case with bile). Changing dressings are also time and labor intensive. Nurses (or other caregivers) have to be meticulously careful when changing dressings with current products on the market because they all involve applications of adhesive directly to the skin, which is time-consuming and laborious to remove.

The proposed apparatus and method allows for skin to be protected from repeated lifting and application of adhesive, as well as protect against any excess fluids that might leak from the site of the catheter. If there is existing skin irritation, wound care can also be applied and sealed within to aid in the healing process. In one embodiment, the apparatus comprises the application of a “foundation” layer of a thin layer of adhesive with a cut-out hole and a slit to go around the drain site. This layer is hypoallergenic, breathable, and waterproof and is intended to adhere to the skin for a long duration and a “dressing” layer or PAP-B that will adhere to the foundational layer for every dressing change. The PAP-B is intended to be changed as often as needed and will be perforated for ease of change. Current wound dressings contain an adhesive over the entire surface of a bottom side applied to the patient's skin. Removal of this excessive adhesive is a general pain point during the dressing change process. The adhesive frame of the PAP-B remaining adhered on top the foundation layer, while the inner absorption portion of the PAP-B is easily perforated for removal, thereby enabling caretakers and practitioners to easily puncture the dressing and remove the soiled dressing. New dressings can be re-applied over the surface of the intact adhesive frame remaining intact on top of the foundation layer, which will not require changing unless there is visible signs of skin irritation. This will preserve the integrity of the skin, reducing irritation while making the process more efficient for nurses and caregivers.

In another preferred embodiment, the adhesive frame of the PAP-B may form a sufficiently stable adhesive contact directly with the patients skin, thereby negating the need for the initial foundation layer. In this “one-layer” embodiment, the PAP-B may have a wider or larger adhesive frame in comparison to the adhesive frame of the PAP-B of the two-layer system. The inner absorption portion may be perforated for removal in similar fashion as with the two-layer system—pulling a tab in any direction along a perforated border.

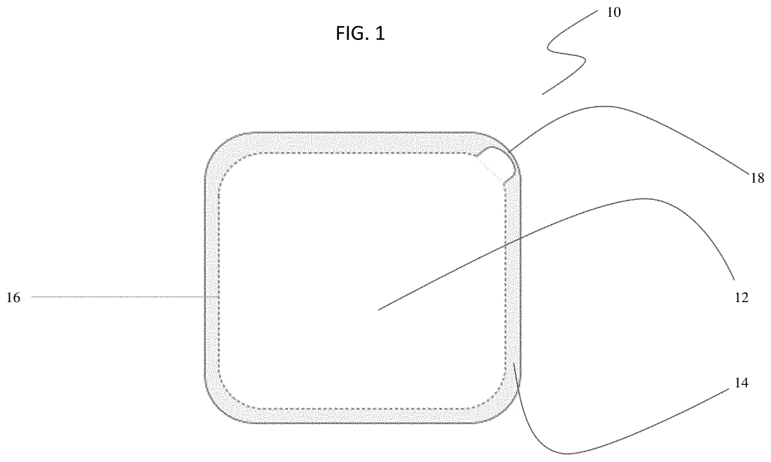

FIG. 1 depicts a top-down view of the of the adhesive frame perforably coupled to the central absorbent portion in accordance with an aspect of the invention. An apparatus for removing a dressing without removing an adhesive portion (PAP-B) 10 is shown. The apparatus comprises: an absorbent central portion 12; an adhesive frame 14; said absorbent portion 12 coupled to the adhesive frame 14 by a perforated border 16; and said perforated border 16 with a tab 18, whereby a user holds the tab 18 and pulls in any one of a direction to loosen free the absorbent portion 12 from the adhesive frame 14 via a perforated border 16, thereby freeing the absorbent portion 12 from an applied skin surface without a need for removing the adhesive frame 14 from the applied skin surface.

While the size of the PAP-B 10 is difficult to ascertain from FIG. 1, in a preferred embodiment, it will conform in size to a standard bandage (3 inches (7.6 cm) by 3 inches (7.6 cm). Smaller and larger variations may be provided for, and different shapes may also be provided for. While shown as substantially square, the PAP-B 10 may be round or oval-shaped, as to cover things like pimples or blisters, for instance, or they can be winged, as might be useful on an elbow or knee. Patterns, designs, and colors may also be provided for, particularly in PAP-B's 10 geared to children.

In a preferred embodiment, latex may often be the most common material of the adhesive frame 14, but plastic, silicate, silicate silicone, polyethylene, or polyurethane may also be used to serve as the material of the adhesive frame 14. The critical feature of the material is that it must be comprised of a moisture-permeable material to protect against exudate strike-through, while still being semi-porous and breathable enough to mitigate the risk of maceration. Moreover, the top surface of the adhesive frame 14 does not comprise an adhesive, with only the bottom surface needing to adhere to a skin or foundation surface. Adhesion may be achieved by any one of an adhesive with a coefficient of adhesion suitable for causing stable contact with a skin surface, while being easily removable from the skin-contact surface. Coefficient of adhesion is a reference to the degree of “stickiness”; the lower the coefficient implying a lower level of “stickiness” between the bottom surface of the adhesive frame with an applied surface (patients skin or top surface of a foundation layer). Examples may include an acrylate, vinyl resin, methacrylate, or epoxy diacrylate-based adhesive. Finally, in a preferred embodiment, the adhesive frame is conformable and water-resistant.

In continuing reference to FIG. 1, the central absorbent portion 12 may be at least one of a soft foam or pad comprised of at least one of a cotton, gel-converting polymer, hydrophilic polyurethane, silicone foam, or absorbent foam with a high moisture transmission rate. Moreover, the central absorbent portion 12 may additionally be coated or embedded with any type of hypoallergenic or anti-bacterial coating to further prevent the risk of contact dermatitis or any other skin condition. In one embodiment, the central absorbent portion 12 may be embedded or coated with medication with known therapeutic value.

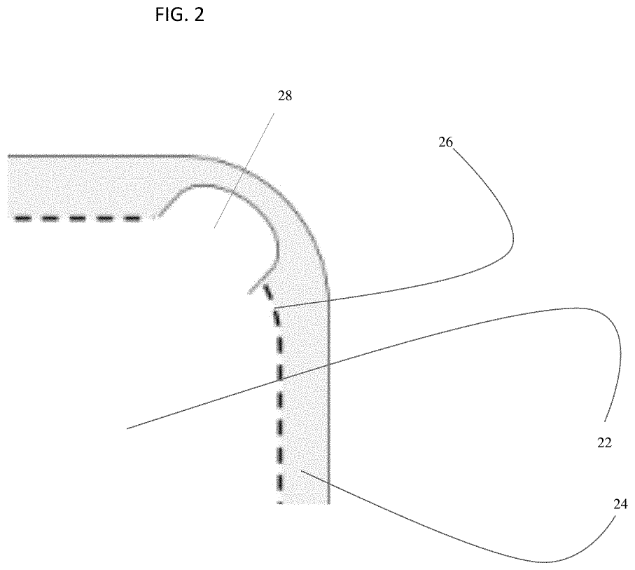

FIG. 2 depicts a close-up view of the perforated border and tab in accordance with an aspect of the invention. As one can appreciate, the perforated border 26 enables performable removal of the absorbent portion 22 from the adhesive frame 24. This perforated solution for removing the soiled absorbent portion 22 avoids constant removal of the adhesive frame 24 from a skin contact surface, thereby mitigating the risk of skin rash, irritation, or medical adhesive related skin injury (MARSI). As shown in FIG. 2, a tab 28 in the right corner allows for the perforated removal of the central absorbent portion 22 from the adhesive frame 24 along a perforated border 26. As shown, the tab 28—border 26 is configured to enable bilateral perforation along the border 26 for removal of the absorbent portion 22. In another embodiment, the tab may be loose or hanging tab or thread enabling perforation in any direction along the perforated border. In yet other embodiments, the tab or thread may be loose or hanging, and enable perforation in any direction—one direction at a time along the entire edge or perimeter of the border. Additionally, there may be a plurality of tabs in any a plurality of directions along the perforated border for ease of removal.

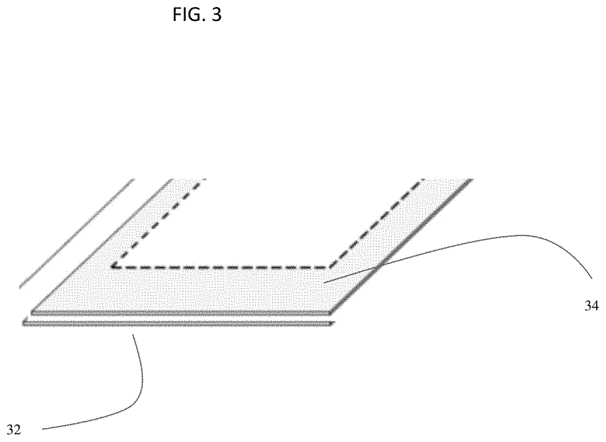

After perforated removal of the soiled absorbent portion, the next PAP-B may be adhered to the previously existing adhesive frame 32. The next PAP-B adhesive frame 34 stacked on top of the previously existing adhesive frame 32 (Shown in FIG. 3—depicting a side view of a plurality of adhesive frames stacked on top of one another in accordance with an aspect of the invention). Any number of adhesive frames may be stacked, depending on the rate of soiling and the frequency of removals of the absorbent portion.

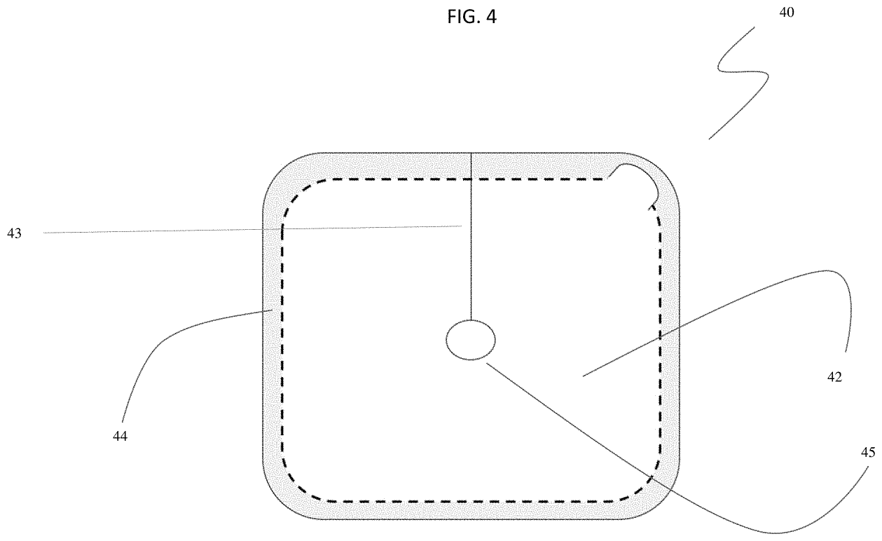

FIG. 4 depicts a top-down view of the adhesive frame coupled to the absorbent portion with a slit opening terminating in a central aperture to accommodate a tube/insertion in accordance with an aspect of the invention. In a preferred embodiment, the PAP-B 40 is disposed with a slit 43 opening along any point of a perimeter of the adhesive frame 44, running laterally through the adhesive frame 44 and central absorbent portion 42, and terminating in a central tube insertion aperture 45 for applying the adhesive frame 44 coupled to the absorbent portion 42 around a tube/insertion.

As shown in FIG. 4, the slit opening 43 runs laterally from a top edge through the adhesive frame 44, absorbent portion 42, and eventually terminating into the central tube insertion aperture 45. However, in other embodiments, the slit may run laterally from any edge of the PAP-B—top edge, side edges, or bottom edge. In yet other embodiments, the slit may run laterally from any of the corner edges of the PAP-B.

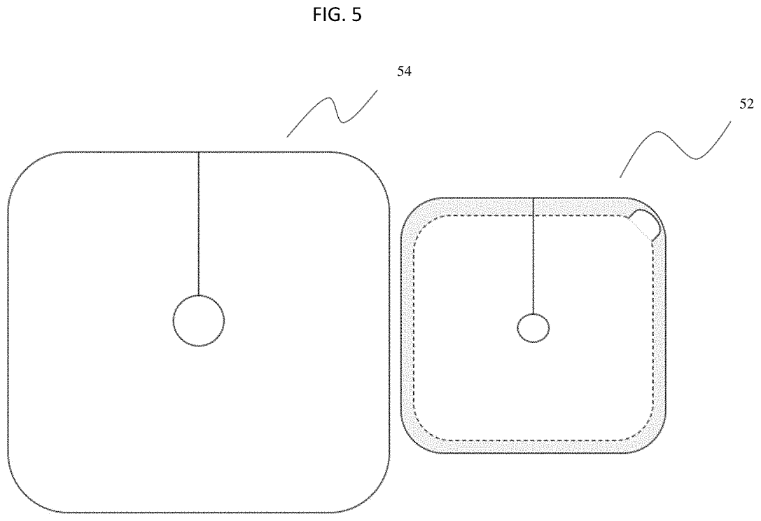

FIG. 5 depicts a top-down view of the adhesive frame coupled to the absorbent portion along side a foundation layer in accordance with an aspect of the invention. The foundation layer 54 may have a similar shape as the PAP-B 52, with a larger dimension so that the PAP-B 52 may adhere above a top surface of the foundation layer 54. The foundation layer may have any sized or dimensioned frame or inner opening to accommodate a tube insertion or wound/area of treatment. While not shown, the bottom surface of the foundation layer 54 is further comprised of any one of an adhesive material with a higher coefficient of adhesion than the adhesive material comprised on the bottom surface of the adhesive frame of the PAP-B 52 in order to be secure in place and peel off the adhesive frame of the PAP-B 52 from the foundation layer 54 without causing dislodgement, tearing, or irritation of the applied skin surface. The material of the foundation layer 54 may be similar as that of the adhesive frame of the PAP-B 52. Additionally, the adhesive material on the bottom surface of the foundation layer may be similar as that of the adhesive material on the bottom surface of the adhesive frame of the PAP-B 52. While the adhesive material may be comprised of the same chemical composition, in some embodiments, the coefficient of adhesion of the adhesive material on the bottom surface of the foundation layer 54 may be lower than that of the adhesive frame of the PAP-B 52. In other embodiments, the adhesive material is exactly identical between the adhesive frame of the PAP-B 52 and the foundation layer 54—both in terms of chemical composition and coefficient of adhesion.

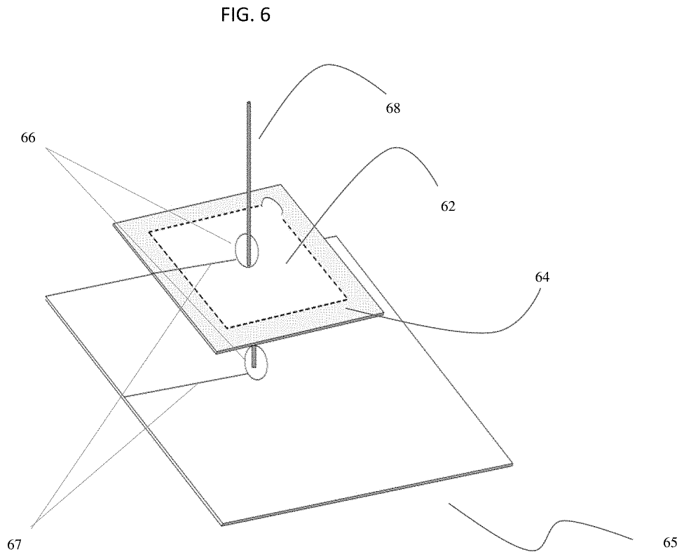

FIG. 6 depicts a top-down view of the adhesive frame coupled to the absorbent portion adhered onto a foundation layer in accordance with an aspect of the invention. FIG. 6 illustrates an apparatus for dressing a wound, said apparatus comprising: an absorbent central portion 62; an adhesive frame 64; and the absorbent central portion 62 perforably coupled to the adhesive frame 64, wherein the adhesive frame 64 is further adhered to any one of a shaped foundation layer 65 with a central opening 66 therebetween, wherein a bottom surface of the adhesive frame 64 is adhered to a top surface of the shaped foundation layer 65 and a bottom surface of the shaped foundation layer 65 is applied to and adhered onto an applied skin surface.

As shown, the foundation layer 65 is disposed with a slit 67 opening along any point of a perimeter of the foundation layer 65, running laterally through the foundation layer 65, and terminating in the central opening 66 to accommodate the tube/insertion 68. As shown in FIG. 6, the slit 67 opening additionally runs laterally from a bottom edge of the PAP-B, through the adhesive frame 64, absorbent portion 62, and eventually terminating into the central opening 66. However, in other embodiments, the slit may run laterally from any edge of the foundation layer or PAP-B—top edge, side edges, or bottom edge. In yet other embodiments, the slit may run laterally from any of the corner edges of the foundation layer or PAP-B.

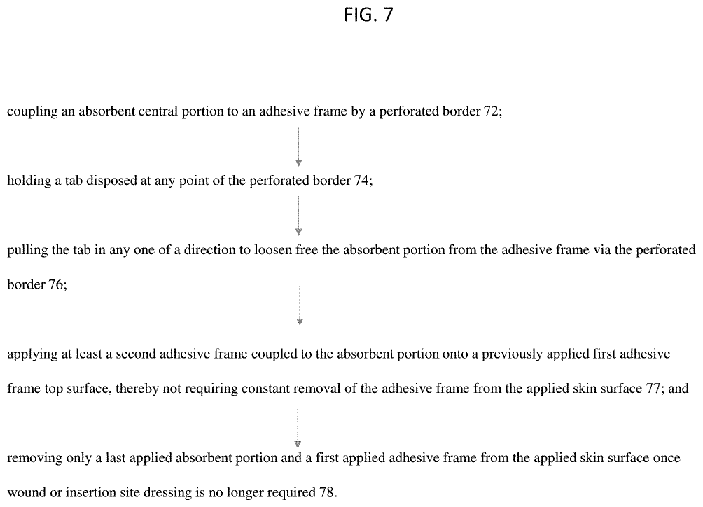

FIG. 7 depicts a method flow diagram in accordance with aspects of the invention. Shown is a method for removing a dressing without removing an adhesive portion, said method comprising the steps of: (1) coupling an absorbent central portion to an adhesive frame by a perforated border 72; (2) holding a tab disposed at any point of the perforated border 74; (3) pulling the tab in any one of a direction to loosen free the absorbent portion from the adhesive frame via the perforated border 76; (4) applying at least a second adhesive frame coupled to the absorbent portion onto a previously applied first adhesive frame top surface, thereby not requiring constant removal of the adhesive frame from the applied skin surface 77; and (5) removing only a last applied absorbent portion and a first applied adhesive frame from the applied skin surface once wound or insertion site dressing is no longer required 78.

The foregoing descriptions of specific embodiments of the invention have been presented for purposes of illustration and description. They are not intended to be exhaustive or to limit the invention to the precise forms disclosed. Modifications and variations are possible in light of the above teaching. The embodiments were chosen and described in order to explain the principles and the application of the invention, thereby enabling others skilled in the art to utilize the invention in its various embodiments and modifications according to the particular purpose contemplated. The scope of the invention is intended to be defined by the claims appended hereto and their equivalents.

What is claimed is:

1. An apparatus for removing a dressing without removing an adhesive portion, said apparatus comprising:
an absorbent central portion;
an adhesive frame;
said absorbent portion coupled to the adhesive frame by a perforated border; and
said perforated border with a tab, whereby a user holds the tab and pulls in any one of a direction to loosen free the absorbent portion from the adhesive frame via the perforated border, thereby freeing the absorbent portion from an applied skin surface without a need for removing the adhesive frame from the applied skin surface.

2. The apparatus of claim 1, wherein the absorbent central portion is at least one of a soft foam or pad comprised of at least one of a gel-converting polymer, hydrophilic polyurethane, silicone foam, or absorbent foam with a high moisture transmission rate.

3. The apparatus of claim 1, wherein the adhesive frame is comprised of any one of a conformable, water-resistant top surface and bottom surface, wherein the top surface is non-adhesive and a bottom surface is adhesive and makes contact with the applied skin surface.

4. The apparatus of claim 3, wherein the adhesive frame is comprised of a moisture-permeable material to protect against exudate strike-through.

5. The apparatus of claim 3, wherein the adhesive frame is comprised of semi-porous, breathable, and water resistant material comprised of at least one of a silicate, silicate silicone, polyethylene, polyurethane, woven fabric, or latex to mitigate a risk of maceration.

6. The apparatus of claim 4, wherein the bottom surface is comprised of any one of an adhesive material with a low coefficient of adhesion in order to be secure in place and easily peeled off the applied skin surface.

7. The apparatus of claim 6, wherein the adhesive material on the bottom surface of the adhesive frame is at least one of an acrylate or vinyl resin.

8. The apparatus of claim 7, wherein the adhesive material is at least one of a methacrylate or epoxy diacrylate.

9. The apparatus of claim 7, further comprising at least one of an hypoallergenic or anti-bacterial coating or embedding to mitigate the risk of contact dermatitis or any skin infection.

10. The apparatus of claim 1, wherein the perforated border enables perforated removal of the absorbent portion from the adhesive frame by avoiding constant removal of the adhesive frame and mitigating the risk of medical adhesive related skin injury (MARSI).

11. The apparatus of claim 1, wherein the tab of the perforated border enables bilateral perforation along the edge or perimeter of the perforated border for removal of the absorbent portion.

12. The apparatus of claim 1, further comprising applying at least a second adhesive frame perforably coupled to the absorbent central portion, wherein the applied surface is a previously applied adhesive frame top surface and not an initially applied skin surface or any skin surface.

13. The apparatus of claim 1, wherein the adhesive frame perforably coupled to the absorbent portion is disposed with a slit opening along any point of a perimeter of the adhesive frame, running laterally through the adhesive frame and central absorbent portion, and terminating in a central tube insertion aperture for applying the adhesive frame coupled to the absorbent portion around a tube insertion site.

14. An apparatus for dressing a wound, said apparatus comprising:
an absorbent central portion;
an adhesive frame; and
the absorbent central portion perforably coupled to the adhesive frame, wherein the adhesive frame is further adhered to any one of a shaped foundation layer with a central opening therebetween, wherein a bottom surface of the adhesive frame is adhered to a top surface of the shaped foundation and a bottom surface of the shaped foundation is applied to and adhered onto an applied skin surface.

15. The apparatus of claim 14, wherein the perforated border comprises a tab, whereby a user holds the tab and pulls in any one of a direction to loosen free the absorbent portion from the adhesive frame via a perforated border, thereby freeing the absorbent portion from an applied skin surface without a need for removing the adhesive frame from the applied skin surface.

16. The apparatus of claim 15, wherein the tab of the perforated border is configured to be held and pulled in a bi-directional manner along a perimeter or edge of the perforated border.

17. The apparatus of claim 14, wherein the absorbent central portion is at least one of a soft foam or pad comprised of at least one of a gel-converting polymer, hydrophilic polyurethane, silicone foam, or absorbent foam with a high moisture transmission rate.

18. The apparatus of claim 14, wherein the adhesive frame is comprised of any one of a conformable, water-resistant top surface and bottom surface, wherein the top surface is non-adhesive and a bottom surface is adhesive and makes contact with the applied skin surface.

19. The apparatus of claim 14, wherein the adhesive frame is comprised of a moisture-permeable material to protect against exudate strike-through.

20. The apparatus of claim 14, wherein the adhesive frame is comprised of semi-porous, breathable, and water-resistant material comprised of at least one of a silicate, silicate silicone, polyethylene, polyurethane, woven fabric, or latex to mitigate a risk of maceration.

21. The apparatus of claim 14, wherein the bottom surface of the adhesive frame is comprised of any one of an adhesive material with a low coefficient of adhesion in order to be secure in place and peeled off the applied skin surface.

22. The apparatus of claim 21, wherein the adhesive material on the bottom surface of the adhesive frame is at least one of an acrylate or vinyl resin.

23. The apparatus of claim 22, wherein the adhesive material is at least one of a methacrylate or epoxy diacrylate.

24. The apparatus of claim 23, wherein the bottom surface of the foundation layer further is comprised of any one of an adhesive material with a higher coefficient of adhesion than the adhesive material comprised on the bottom surface of the adhesive frame in order to be secure in place and peel off the adhesive frame from the foundation layer without causing dislodgement, tearing, or irritation of the applied skin surface.

25. The apparatus of claim 24, wherein the adhesive material on the bottom surface of the foundation layer is at least one of an acrylate or vinyl resin.

26. The apparatus of claim 25, further comprising at least one of an hypoallergenic or an anti-bacterial coating or embedding to mitigate the risk of contact dermatitis or any skin infection.

27. The apparatus of claim 14, wherein the perforated border enables perforated removal of the absorbent portion from the adhesive frame by avoiding constant removal of the adhesive frame and mitigating the risk of medical adhesive related skin injury (MARSI).

28. The apparatus of claim 14, further comprising applying at least a second adhesive frame perforably coupled to the absorbent central portion, wherein the applied surface is a previously applied adhesive frame top surface and not an initially applied skin surface or any skin surface.

29. The apparatus of claim 14, wherein the foundation layer is disposed with a slit opening along any point of a perimeter of the foundation layer, running laterally through the foundation layer, and terminating in the central opening to accommodate the tube insertion site.

30. A method for removing a dressing without removing an adhesive portion, said method comprising the steps of:
coupling an absorbent central portion to an adhesive frame by a perforated border;
holding a tab disposed at any point of the perforated border;
pulling the tab in any one of a direction to loosen free the absorbent portion from the adhesive frame via the perforated border;
applying at least a second adhesive frame coupled to the absorbent portion onto a previously applied first adhesive frame top surface, thereby not requiring constant removal of the adhesive frame from the applied skin surface; and
removing only a last applied absorbent portion and a first applied adhesive frame from the applied skin surface once wound or insertion site dressing is no longer required.
